(19) United States
(12) Patent Application Publication
(10) Pub. No.: US 2026/0345843 A1
(43) Pub. Date: Nov. 5, 2026

(54) SPATIAL UNIT-BASED MULTIMODAL INFECTION CONTROL SYSTEM AND METHOD FOR MEDICAL FACILITY

(71) Applicants: SEOUL NATIONAL UNIVERSITY HOSPITAL, Seoul (KR); SEOUL NATIONAL UNIVERSITY R&DB FOUNDATION, Seoul (KR)

(72) Inventors: Sungwan KIM, Yongin-si (KR); Sang Do SHIN, Seoul (KR); Hangman ZO, Seoul (KR); Myoungsouk YEO, Seoul (KR); Kihong KIM, Seoul (KR); Changhoon BAEK, Seoul (KR); Min Hyuk LIM, Seoul (KR); Minwoo CHO, Seoul (KR); Jaewoo SHIM, Seoul (KR); Dong Hyun CHOI, Seoul (KR); Sungwoo PARK, Seoul (KR); Jonghyeon LEE, Seoul (KR); Byoungjun JEON, Seoul (KR); Dan YOON, Seoul (KR); Byeongsoo KIM, Seoul (KR); Young Gyun KIM, Busan (KR)

(73) Assignees: SEOUL NATIONAL UNIVERSITY HOSPITAL, Seoul (KR); SEOUL NATIONAL UNIVERSITY R&DB FOUNDATION, Seoul (KR)

(21) Appl. No.: 19/198,565

(22) Filed: May 5, 2025

(51) Int. Cl. A61L 2/10; A61L 103/15

(52) U.S. Cl. CPC A61L 2/10; A61L 2103/15; A61L 2202/122

(57) ABSTRACT

According to the present disclosure, there are disclosed a spatial unit-based multimodal infection control system and method for a medical facility. The spatial unit-based multimodal infection control system includes: a protection module including walls configured to form an isolation space, and configured to provide a passage for medical treatment; a circulation module disposed in an intake that is formed in the ceiling surface of the walls, and configured to perform ventilation and air-conditioning functions in a circulation mode; a disinfection module including at least one disinfection source placed on the inner wall surface or ceiling surface of the walls, and configured to disinfect the inside of the isolation space in a disinfection mode; and a control module configured to control the disinfection module and the circulation module so that the disinfection mode and the circulation mode are turned on in a standby state.

### BACKGROUND

### 1. Technical Field

The present disclosure relates to a spatial unit-based multimodal infection control system and method for a medical facility, and more specifically, to a system capable of providing modular isolation spaces in order to efficiently block the spread of infection.

Information about the research and development project that supported the present invention is as follows:
Unique Project Number: RS-2025-02222290
Department Name: Ministry of Health and Welfare, Korea Disease Control and Prevention Agency
Research Management Organization: Korea Health Industry Development Institute
Research Project Name: Research and Development for Enhancing Infectious Disease Response Capacity in Medical & Healthcare Settings
Research Task Name: Optimization and commercialization of Multi-modality based Infection Control Bed Isolation System (MICBIS)
Organization in Charge: Seoul National University Hospital
Research Period: Apr. 1, 2025 to Dec. 31, 2026
Contribution Rate: 1/2
Unique Project Number: RS-2022-KH124656
Department Name: Ministry of Health and Welfare
Research Management Organization: Korea Health Industry Development Institute
Research Project Name: Development of Technologies for Enhancing Medical Safety Against Infectious Diseases
Research Task Name: Development of Novel DIsinfection Reinforcement Technology based Emergency Department by multimodality infection control system optimized for emergency care
Organization in Charge: Seoul National University Hospital
Research Period: Apr. 1, 2022 to Dec. 31, 2024
Contribution Rate: 1/2

### 2. Description of the Related Art

According to the announcement by the National Emergency Medical Center in Korea (in 2021), approximately 8 million emergency patients visited local emergency rooms annually.

Emergency patients visit without reservations to receive rapid evaluation and treatment for internal medical symptoms or injuries that suddenly occur, and emergency treatment begins without final diagnoses. Accordingly, it is difficult to check whether there is an infectious disease having a risk of spread before a detailed examination.

The emergency rooms of general hospitals in Korea are chronically overcrowded due to the large numbers of patients and the lack of hospital beds compared to their size. Furthermore, as the lengths of time for which patients stay in emergency rooms increase, patients with weakened immune systems are bound to become vulnerable to infection.

Since the outbreak of the Covid-19 infectious disease disaster, hospitals have been providing normal treatment after checking the results of detailed examinations for all patients suspected of having infectious disease. However, due to the lack of hospital beds and the low patient accommodation capacity of hospitals, emergency the emergency rooms of higher-level treatment for all emergency patients is severely limited.

As for the path of an infected patient in a typical emergency room, when an infection occurs, an infected patient is moved according to the process of patient transfer, emergency room visit and waiting, emergency care and treatment, diagnosis, and isolation. In this case, when a patient uses public transportation during a patient transfer process, the probability of contact with the general public increases, which increases the risk of infection. Furthermore, since the average stay in the emergency room is seven hours, the probability of contact with medical staff and general patients before diagnosis increases, which increases the risk of infection. Moreover, the possibility of infection increases due to the entering and leaving of medical staff for treatment after diagnosis.

Since it is impossible to completely screen for infectious diseases only based on triage assessment without detailed examinations, infection may be spread by infected patients who are not preemptively isolated, which may lead to the suspension of the overall emergency room operation. Accordingly, it is necessary to develop technology that can effectively block the spread of infection in an emergency room.

In hospitals, during the process of diagnosing and isolating infected patients, the characteristics of patients accommodated, the risks of infection, the levels of patient condition monitoring required, the levels of treatment and care to be provided, the number of patients staying per area, and the frequency of contact between medical staff and patients are identified, and then the isolation of patients is performed on a per-spatial unit basis.

In this manner, emergency medical facilities have different purposes and characteristics for individual spatial units, have different levels of requirements for infection control, and need to provide an environment where all emergency patients can receive treatment safely without the interruption of emergency medical care on a per-spatial unit basis.

Currently, infectious disaster response is based on the isolation of suspected infected patients using negative pressure facilities. However, as the number of suspected infected patients increases or the infectious disaster period becomes longer, limitations arise due to the lack of negative pressure facilities and the operation of negative pressure facilities.

Therefore, there is a demand for the development of an emergency medical facility operation technology that can effectively deal with an infectious disaster by utilizing technology capable of controlling spatial unit-based infection management within an emergency medical facility while overcoming existing limitations.

### PRIOR ART LITERATURE

### Patent Literature

Korean Patent Application Publication No. 10-2021-0154303 (published on Dec. 21, 2021)

### SUMMARY

The present disclosure has been conceived in response to the above-described background art, and an object of the present disclosure is to provide a spatial unit-based multimodal infection control system for a medical facility that provides a modular isolation space including a protection module, a circulation module, and a disinfection module in a medical facility to block physical, chemical, and biological infections, thereby being capable of efficiently blocking the spread of infections.

However, the objects to be achieved in the present disclosure are not limited to the objects mentioned above, and other objects not mentioned may be clearly understood based on the following description.

According to one embodiment of the present disclosure for achieving the above-described object, there is disclosed a spatial unit-based multimodal infection control system for a medical facility. The spatial unit-based multimodal infection control system includes: a protection module including walls configured to form an isolation space, and configured to provide a passage for medical treatment that is disposed in one surface of the walls; a circulation module disposed in an intake that is formed in the ceiling surface of the walls, and configured to perform ventilation and air-conditioning functions in a circulation mode; a disinfection module including at least one disinfection source placed on the inner wall surface or ceiling surface of the walls, and configured to disinfect the inside of the isolation space by using the disinfection source in disinfection mode; and a control module configured to control the disinfection module and the circulation module so that the disinfection mode and the circulation mode are turned on in a standby state in which there is no patient in the isolation space.

Alternatively, the protection module may include: the walls arranged vertically based on a floor surface, and configured to provide the isolation space; a ceiling panel disposed on the ceiling surface located over the walls, and configured to close the isolation space except for the intake; a strip door disposed in the passage; and a window disposed in one surface of the walls located in a direction facing the strip door, and serving as a passage for entry and exit of a wiring means for connection with a medical device or power source provided in a medical facility.

Alternatively, the walls may include: one or more support columns vertically arranged at respective corners of the isolation space; panels arranged between the individual support columns, and forming wall surfaces except for the wall surface in which the strip door is installed; and screen doors configured to be selectively opened and closed in vertical directions along the outer surfaces of the walls by sliding rollers arranged at the top ends of the walls.

Alternatively, the control module may control the screen doors to be closed in order to cover the inside of the isolation space in the disinfection mode.

Alternatively, the circulation module may include: a fan filter unit configured to perform internal ventilation and air-conditioning functions by blocking the inflow of outside air and the outflow of inside air to and from the isolation space; and an adaptive nozzle disposed to discharge the air flow, formed by the fan filter unit, out of the isolation space, and formed to be rotatable in order to operate in conjunction with an air-conditioning system of the medical facility.

Alternatively, the fan filter unit may include at least one of a HEPA filter, an activated charcoal filter, and a pre-filter.

Alternatively, the disinfection source may include at least one of one or more ultraviolet radiation means and one or more disinfectant spray means.

Alternatively, the installation locations and number of the ultraviolet radiation means may be set based on a predicted time-based effective disinfection range, and the time-based effective disinfection range in which the isolation space is disinfected is predicted based on the ultraviolet intensity by means of the ultraviolet radiation means and the operation time information of the disinfection mode.

Alternatively, the time-based effective disinfection range may be calculated as a time-based effective disinfection range based on the installation locations and number of the ultraviolet radiation means according to the simulation using a multiple points source summation (MPSS) model in the case where there is an object inside the isolation space.

Alternatively, the ultraviolet intensity (It) by means of the MPSS model may be calculated by Equation 1 below using Snell's law and a Fresnel coefficient, and the ultraviolet intensity by means of n ultraviolet radiation means may be calculated by summing all ultraviolet intensities by means of the respective ultraviolet radiation means disposed at different installation locations:

$${I}_{i} = ( 1 - R ) \times ( 1 - R ) \times \frac{P / n}{4 {π ( {d}_{1} + {d}_{2} + {d}_{3} )}^{2}} \times {e}^{- α {d}_{2}} \qquad ( 1 )$$

P: length of the ultraviolet radiation means
n: permittivity
d₁, d₂, and d₃: distances to points where the ultraviolet intensities from the individual ultraviolet radiation means are measured, respectively

$$Fresnel coefficient : {R}_{ij} = \frac{1}{2} \{ {( \frac{{n}_{j} sin {θ}_{i} - {n}_{i} cos {θ}_{j}}{{n}_{i} sin {θ}_{j} + {n}_{j} cos {θ}_{i}} )}^{2} + {( \frac{{n}_{i} sin {θ}_{i} - {n}_{j} cos {θ}_{j}}{{n}_{i} sin {θ}_{i} + {n}_{j} cos {θ}_{j}} )}^{2} \}$$

Snell's law: n₁ sin θ₁=n₂ sin θ₂
e: permeability

Alternatively, the ultraviolet radiation means may include at least one ultraviolet radiation means installed at each corner of the isolation space, or at least one ultraviolet radiation means installed on an edge side of the ceiling surface.

Alternatively, the spatial unit-based multimodal infection control system may further include a motion detection means disposed on one side of the passage, and the control module may control the ON/OFF operation of the disinfection mode or the ON/OFF operation of a lighting device disposed in the protection module based on the detection result of the motion detection means.

According to one embodiment of the present disclosure, there is disclosed a spatial unit-based multimodal infection control method for a medical facility that is performed by a system including at least one processor. The spatial unit-based multimodal infection control method includes: turning on a disinfection mode that performs disinfection by means of a disinfection source for a preset operation time in a standby state in which there is no patient inside a protection module that forms an isolation space by using walls; turning off the disinfection mode after the operation time has elapsed in the standby state, and turning on a first circulation mode that removes the chemical debris formed by the disinfection mode; and turning off the disinfection mode and turning on a second circulation mode that performs ventilation and air conditioning functions for the isolation space in an isolation state in which there is a patient in the isolation space. The disinfection source includes at least one of one or more ultraviolet irradiation means and one or more disinfectant spray means.

Alternatively, the output of a circulation module in the first circulation mode may be set to be higher than that of the circulation module in the second circulation mode.

The system according to one embodiment of the present disclosure may provide a modular isolation space that protects infected patients, their guardians, or medical staff from infection, performs ventilation and the blocking of air in the isolation space, and performs disinfection in a standby state in which there is not patient in the isolation space, in consideration of the installation environment or conditions of a medical facility, thereby providing the effect of enabling multiple infection control.

In addition, the system according to one embodiment of the present disclosure may provide standard facility specifications that considers a required infection control level for each spatial unit of an emergency medical facility based on the results of the analysis of infectious agent spread, thereby providing the effect of maximizing the efficiency of infection control while maintaining the existing level of treatment function for an emergency patient or an infected patient.

In this manner, the system according to one embodiment of the present disclosure may provide the effect of, based on a multimodal infection control technology capable of controlling physical, chemical, biological and infections, effectively blocking the spread of infection and also establishing an infection control system for efficient spatial unit-based infection control within a medical facility.

### BRIEF DESCRIPTION OF THE DRAWINGS

The above and other objects, features, and advantages of the present invention will be more clearly understood from the following detailed description taken in conjunction with the accompanying drawings, in which:

FIG. 1 is a diagram illustrating the configuration of a spatial unit-based multimodal infection control system for a medical facility according to one embodiment of the present disclosure;

FIGS. 2A and 2B are diagrams illustrating an actual implementation of a spatial unit-based multimodal infection control system for a medical facility according to one embodiment of the present disclosure;

FIG. 3 is a drawing illustrating the configuration of a circulation module according to one embodiment of the present disclosure;

FIG. 4 is a block diagram of a control module according to one embodiment of the present disclosure;

FIG. 5 is a diagram illustrating an infectious agent spread analysis process for selecting the location of an intake according to one embodiment of the present disclosure;

FIGS. 6A and 6B are diagrams illustrating the results of the analysis of a respiratory model through CFD modeling in the infectious agent spread analysis process of FIG. 5;

FIGS. 7A and 7B are diagrams illustrating the results of the infectious agent spread analysis of FIG. 5;

FIG. 8 shows diagrams illustrating an effective disinfection range in a disinfection mode according to one embodiment of the present disclosure;

FIG. 9A and FIG. 9B show diagrams illustrating simulation results predicting a time-based disinfection range by means of one ultraviolet radiation means according to one embodiment of the present disclosure;

FIG. 10A and FIG. 10B show diagrams illustrating simulation results predicting a time-based disinfection range by means of two ultraviolet radiation means according to one embodiment of the present disclosure;

FIG. 11A, FIG. 11B and FIG. 11C show diagrams illustrating simulation results predicting a time-based disinfection range by means of ultraviolet radiation means arranged at various installation locations according to one embodiment of the present disclosure;

FIG. 12A and FIG. 12B show diagrams illustrating an ultraviolet intensity distribution and actually measured ultraviolet intensities according to one embodiment of the present disclosure;

FIGS. 13A and 13B are diagrams illustrating the installation locations of ultraviolet radiation means according to one embodiment of the present disclosure;

FIG. 14 is a flowchart illustrating a spatial unit-based multimodal infection control method for a medical facility according to one embodiment of the present disclosure; and

FIG. 15 is a diagram illustrating the configuration of a spatial unit-based multimodal infection control system for a medical facility according to another embodiment of the present disclosure.

### DETAILED DESCRIPTION

Embodiments of the present disclosure will be described in detail below with reference to the accompanying drawings so that those having ordinary skill in the art of the present disclosure (hereinafter referred to as those skilled in the art) can easily implement the present disclosure. The embodiments presented in the present disclosure are provided to enable those skilled in the art to use or practice the content of the present disclosure. Accordingly, various modifications to embodiments of the present disclosure will be apparent to those skilled in the art. That is, the present disclosure may be implemented in various different forms and is not limited to the following embodiments.

The same or similar reference numerals denote the same or similar components throughout the specification of the present disclosure. Additionally, in order to clearly describe the present disclosure, reference numerals for parts that are not related to the description of the present disclosure may be omitted in the drawings.

The term “or” used herein is intended not to mean an exclusive “or” but to mean an inclusive “or.” That is, unless otherwise specified herein or the meaning is not clear from the context, the clause “X uses A or B” should be understood to mean one of the natural inclusive substitutions. For example, unless otherwise specified herein or the meaning is not clear from the context, the clause “X uses A or B” may be interpreted as any one of a case where X uses A, a case where X uses B, and a case where X uses both A and B.

The term “and/or” used herein should be understood to refer to and include all possible combinations of one or more of listed related concepts.

The terms “include” and/or “including” used herein should be understood to mean that specific features and/or components are present. However, the terms “include” and/or “including” should be understood as not excluding the presence or addition of one or more other features, one or more other components, and/or combinations thereof.

Unless otherwise specified herein or unless the context clearly indicates a singular form, the singular form should generally be construed to include “one or more.”

The term “n-th (n is a natural number)” used herein may be understood as an expression used to distinguish the components of the present disclosure according to a predetermined criterion such as a functional perspective, a structural perspective, or the convenience of description. For example, in the present disclosure, components performing different functional roles may be distinguished as a first component or a second component. However, components that are substantially the same within the technical spirit of the present disclosure but should be distinguished for the convenience of description may also be distinguished as a first component or a second component.

The term “obtaining” used herein may be understood to mean not only receiving data from an external device or a system over a wired/wireless communication network but also generating data in an on-device form.

Meanwhile, the term “module” or “unit” used herein may be understood as a term referring to an independent functional unit processing computing resources, such as a computer-related entity, firmware, software or part thereof, hardware or part thereof, or a combination of software and hardware. In this case, the “module” or “unit” may be a unit composed of a single component, or may be a unit expressed as a combination or set of multiple components. For example, in the narrow sense, the term “module” or “unit” may refer to a hardware component or set of of a computing device, an application program components performing a specific function of software, a procedure implemented through the execution of software, a set of instructions for the execution of a program, or the like. Additionally, in the broad sense, the term “module” or “unit” may refer to a computing device itself constituting part of a system, an application running on the computing device, or the like. However, the above-described concepts are only examples, and the concept of “module” or “unit” may be defined in various manners within a range understandable to those skilled in the art based on the content of the present disclosure.

The term “model” used herein may be understood as a system implemented using mathematical concepts and language to solve a specific problem, a set of software units intended to solve a specific problem, or an abstract model for a process intended to solve a specific problem. For example, a neural network “model” may refer to an overall system implemented as a neural network that is provided with problem-solving capabilities through training. In this case, the neural network may be provided with problem-solving capabilities by optimizing parameters connecting nodes or neurons through training. The neural network “model” may include a single neural network, or a neural network set in which multiple neural networks are combined together.

The term “block” used herein may be understood as a set of components that are classified based on various criteria such as type, function, and/or the like. Accordingly, the components classified as one “block” may be changed in various manners depending on the criteria. For example, a neural network “block” may be understood as a set of neural networks that include at least one neural network. In this case, it may be assumed that the neural networks included in the neural network “block” perform specific operations in the same manner.

The foregoing descriptions of the terms are intended to help to understand the present disclosure. Accordingly, it should be noted that unless the above-described terms are explicitly described as limiting the content of the present disclosure, they are not used in the sense of limiting the technical spirit of the present disclosure.

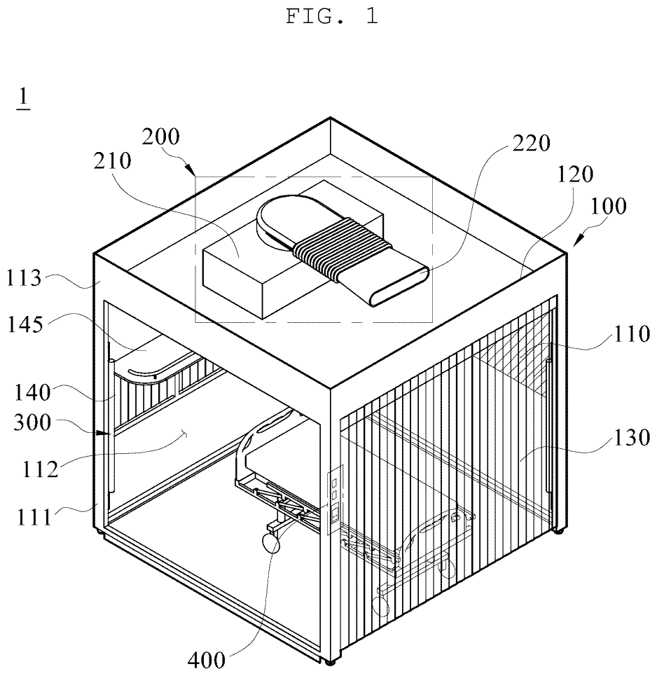

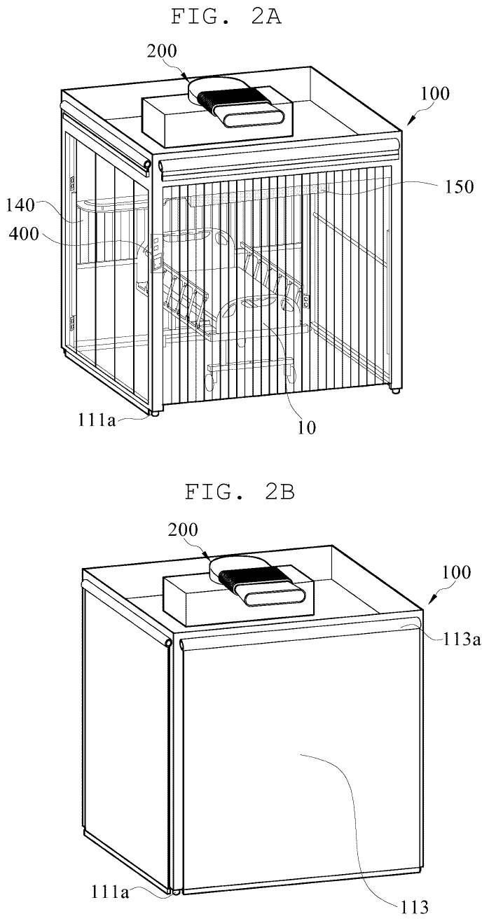

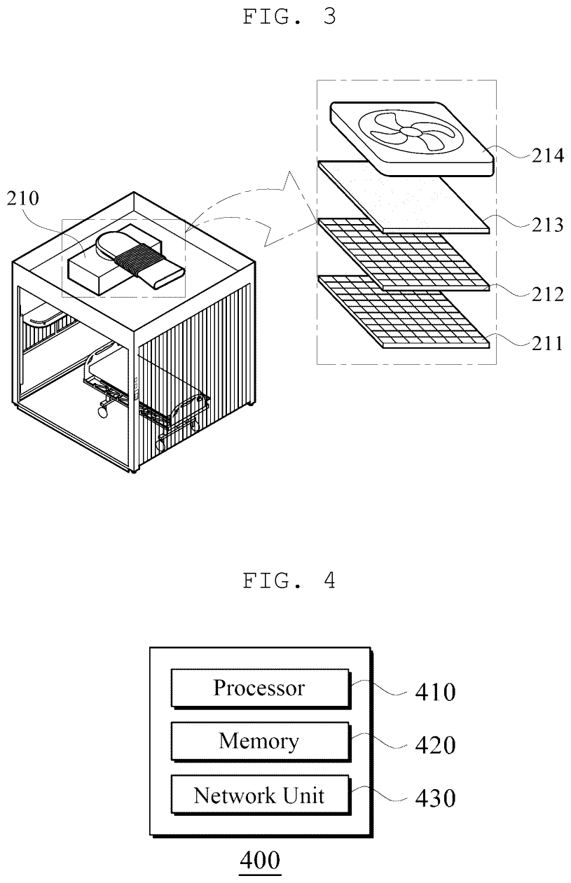

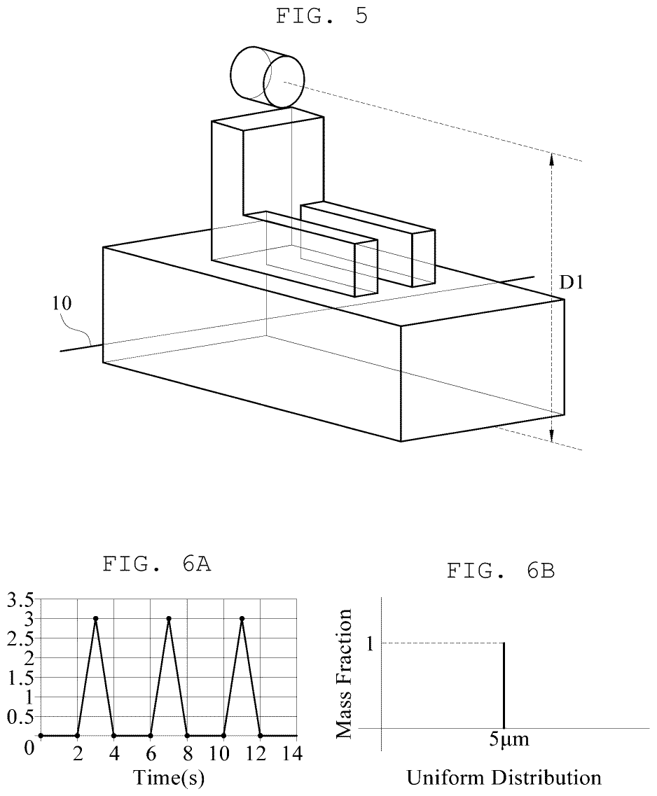

FIG. 1 is a diagram illustrating the configuration of a spatial unit-based multimodal infection control system for a medical facility according to one embodiment of the present disclosure, FIG. 2 is a illustrating an actual diagram implementation of a spatial unit-based multimodal infection control system for a medical facility according to one embodiment of the present disclosure, and FIG. 3 is a drawing illustrating the configuration of a circulation module according to one embodiment of the present disclosure.

Referring to FIG. 1, a spatial unit-based multimodal infection control system 1 for a medical facility includes, but is not limited to, a protection module 100, a circulation module 200, a disinfection module 300, and a control module 400.

The protection module 100 includes walls 110 forming an isolation space, and provides a passage for medical treatment that is disposed in one surface of the walls 110. The protection module 100 includes the walls 110 arranged vertically based on a floor surface and configured to provide the isolation space, a ceiling panel 120 disposed on a ceiling surface located over the walls 110 and configured to close the isolation space except for an intake 121, a strip door 130 disposed in the passage, a window 140 disposed in one surface of the walls 110 located in a direction facing the strip door 130 and serving as a passage for the entry and exit of a wiring means for connection with a medical device or power source provided in a medical facility, and at least one lighting device 150 disposed on the ceiling surface or a corner of the walls 110 and configured to light the inside of the isolation space.

The window 140 has a shelf 145 disposed along its top end surface, the shelf 145 has a protruding surface protruding into the inside of the isolation space, and the protruding surface may have a flat, curved, or sloping surface. This window 140 may be constructed in the form of a strip door, or may be constructed in the form of a door that can be opened and closed in the vertical direction or in the lateral direction. This window 140 is closed in a standby state without a patient, but is opened in an isolation state with a patient and serves as a passage for allowing a connection pipe for the combination between an external medical device and a medical device inside the isolation space or the like to enter and exit via an open space and for allowing a wiring means such as a cable for the connection of another medical device or electronic device to a power source or the like to enter and exit.

Meanwhile, the walls 110 may include one or more support columns 111 vertically arranged at respective corners of the isolation space, panels 112 arranged between the individual support columns 111 and forming wall surfaces, and screen doors 113 configured to be opened and closed in the vertical directions along the outer surfaces of the walls 110 by sliding rollers 113a arranged at the top ends of the walls 110. As shown in FIG. 2, the walls 110 have the transparent panels 112 formed of soft walls, and the screen doors 113 may serve as screens for protecting a patient's privacy and blocking ultraviolet rays. The screen doors 113 may be closed to cover the inside of the isolation space when a disinfection mode is turned on, and may be opened when the disinfection mode is turned off.

The protection module 100 may include wheels 111a arranged on the lowest end surfaces of the support columns 111, the isolation space formed by the protection module 100 may be moved using the wheels 111a, and the protection module 100 may include wheel stoppers (not shown) configured to stop the rotation of the wheels 111a at a stop location.

This protection module 100 may be fabricated such that at least one wall surface can be rapidly and completely opened and closed to allow the use of large equipment in an emergency and such that all possible outside portions thereof except for the intake 121 are sealed to minimize the inflow and outflow of air from and to the outside. Accordingly, a patient, medical staff, and a guardian may be physically blocked from being infected.

The transparent panels 112 allow medical staff to visually check a patient from the outside. In this case, the walls 110 may be made of a chemically resistant material that has an anti-static function and is also resistant to disinfectants such as ethanol, and may be made of a highly durable material that is resistant to impact and wear and does not break.

The circulation module 200 is disposed in the intake 121 formed in the ceiling surface of the walls, and performs ventilation and air-conditioning functions in a circulation mode. This circulation module 200 includes a fan filter unit 210 connected to the intake 121 and configured to perform internal ventilation and air-conditioning functions by blocking the inflow of outside air and the outflow of inside air to and from the isolation space, and an adaptive nozzle 220 disposed to discharge the air flow, formed by the fan filter unit 210, out of the isolation space and formed to be rotatable in order to operate in conjunction with the air-conditioning system of the medical facility.

The adaptive nozzle 220 may be formed to be flexibly bent or be freely stretched or bent at a desired angle by using a corrugated structure in order to maintain air-conditioning performance in various installation environments. The angle and shape of the adaptive nozzle 220 may be determined in accordance with the air-conditioning environment of a medical facility when the spatial unit-based multimodal infection control system 1 for a medical facility is first installed in the medical facility. In other words, the shape of the adaptive nozzle 220 may be adaptively changed in accordance with the environment of the medical facility where the system 1 is installed.

As shown in FIG. 3, by considering noise and the suppression of infectious agents, the fan filter unit 210 may include a fan assembly 214 equipped with a blower fan, and air filters 211, 212, and 213 arranged behind the fan assembly 214. In this case, the air filters 211, 212, and 213 may additionally employ an activated charcoal filter configured to remove odor and harmful substances such as formaldehyde or benzene, a pre-filter, or the like, in addition to a HEPA filter configured to remove fine dust, through additional attachment.

The disinfection module 300 has at least one disinfection source placed on the inner wall surface or ceiling surface of the walls 110, and disinfects the inside of the isolation space by using the disinfection source in a disinfection mode. In this case, the disinfection source may employ one or more of an ultraviolet radiation means 310 and a disinfectant spray means (not shown).

An ultraviolet disinfection n method using the ultraviolet radiation means 310 destroys and inactivates the DNA or RNA of microorganisms such as viruses, bacteria, and fungi. Although ultraviolet rays may destroy the DNA or RNA of microorganisms in all wavelength ranges, this ultraviolet radiation means 310 basically employs an ultraviolet light-emitting diode that is environmentally friendly and durable. However, the ultraviolet radiation means 310 is not limited to an ultraviolet light-emitting diode, but may employ a general ultraviolet lamp or plasma lamp.

The ultraviolet radiation means 310 may adjust the ultraviolet wavelength range so that ultraviolet rays can effectively perform disinfection according to the environment of the isolation space. That is, the ultraviolet radiation means 310 may be designed to radiate rays having a single wavelength by using any one of a UV-A wavelength LED, a UV-B wavelength LED, and a UV-C wavelength LED alone, or to radiate rays having mixed wavelengths by using at least two of an UV-A wavelength LED, an UV-B wavelength LED, and a UV-C wavelength LED in combination.

Ultraviolet rays may destroy the or RNA of DNA microorganisms in all wavelength ranges. Among them, a method using ultraviolet rays in the UV-C wavelength range (from 100 nm to 280 nm) having strong disinfection power is referred to as Ultraviolet Germicidal Irradiation (UVGI) or Germicidal Ultraviolet (GUV).

However, UV-C can cause keratitis or conjunctivitis along with pain when it has been in contact with the eyes for a long time, so that it is necessary to limit the use of UV-C indoors in order to prevent an occupant or a patient from being harmed.

Accordingly, the ultraviolet radiation means 310 may be selected by considering the wavelength range where disinfection wavelengths exhibit an excellent disinfection effect, data by which disinfection and safety can be verified, such as a certified test report, and an appropriate safety device for exposure to ultraviolet rays.

The disinfectant spray means sprays disinfectant by using a chemical disinfection method such as electrostatic spraying, fogger, dry fogging, or vapor spraying, so that the chemical substance floats in the air for a long time and exhibits a sustained disinfection effect and the disinfectant better attaches to a surface and exhibits an appropriate disinfection effect even in a space having a small exposed surface. This disinfectant spray means may be used in the isolation space of the protection module 100, and may be applied to an automatic recognition disinfection device when the strip door 130 is opened and closed.

The circulation module 200 and the disinfection module 300 may chemically and biologically block the secondary infections of medical staff, transport personnel, and a guardian from an infected patient.

The control module 400 may control the disinfection module 300 or the circulation module 200 so that a disinfection mode and a first circulation mode are turned on in a standby state when there is no patient in the isolation space and only a second circulation mode is turned on in an isolation state when there is a patient in the isolation space. Furthermore, the control module 400 may adjust the amounts of ventilation by controlling the speed of the blower fan to be appropriate for the first circulation mode and the second circulation mode.

When a motion detection means (not shown) is disposed on a passage, the control module 400 may perform the ON/OFF control of the disinfection mode or the ON/OFF control of the lighting device 150 based on the detection result of the motion detection means. For example, when motion is detected by the motion detection means in the standby state, the control module 400 may stop the disinfection mode and turn on the lighting device 150.

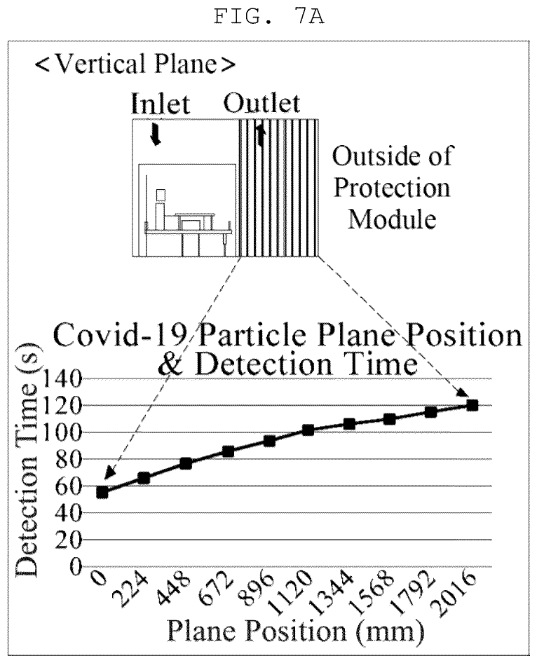

FIG. 4 is a block diagram of a control module according to one embodiment of the present disclosure.

The control module 400 according to one embodiment of the present disclosure may be a hardware device or a part of a hardware device that performs the comprehensive processing and computation of data, or may be a software-based computing environment connected over a communication network. For example, the control module 400 may be a server that is a main agent for performing an intensive data processing function as a computing device and sharing resources, or may be a client that shares resources through interaction with a server. Furthermore, the control module 400 may be a cloud system in which multiple servers and clients comprehensively process data while interacting with each other. Since the above description is only one example related to the type of computing device, the type of computing device may be configured in various manners within a range understandable to those skilled in the art based on the content of the present disclosure.

Referring to FIG. 4, the control module 400 according to one embodiment of the present disclosure may include a processor 410, memory 420, and a network unit 430. However, FIG. 4 shows only an example, and accordingly, the control module 400 may include other components for implementing a computing environment. Furthermore, only some of the components disclosed above may be included in the control module 400.

The processor 410 according to one embodiment of the present disclosure may be understood as a constituent unit including hardware and/or software for performing computing operations. For example, the processor 410 may read a computer program and perform data processing for machine learning. The processor 410 may process operation processes such as the processing of input data for machine learning, the extraction of features for machine learning, and the computation of errors based on backpropagation. The processor 410 for performing such data processing may include a central processing unit (CPU), a general purpose graphics processing unit (GPGPU), a tensor processing unit (TPU), an application specific integrated circuit (ASIC), or a field programmable gate array (FPGA). Since the types of processor 410 described above are only examples, the type of processor 410 may be configured in various manners within a range understandable to those skilled in the art based on the content of the present disclosure.

The processor 410 may turn on a disinfection mode and a circulation mode in a standby state, and may maintain the air-conditioning performance of various installation environments through cooperative operation with the air-conditioning system of the medical facility via fan speed control during a circulation mode operation. This processor 410 may apply multimodal infection control technology to a simple isolation facility or clean room including the protection module 100, the circulation module 200, and the disinfection module 300, may minimize interference with medical procedures in various modes according to user scenarios, and may provide an easily usable interface.

The memory 420 according to one embodiment of the present disclosure may be understood as a constituent unit including hardware and/or software for storing and managing data that is processed in the computing device 100. That is, the memory 420 may store any type of data generated or determined by the processor 410 and any type of data received by the network unit 130. For example, the memory 420 may include at least one type of storage medium of a flash memory type, hard disk type, multimedia card micro type, and card type memory, random access memory (RAM), static random access memory (SRAM), read-only memory (ROM), electrically erasable programmable read-only memory (EEPROM), programmable read-only memory (PROM), magnetic memory, a magnetic disk, and an optical disk. Furthermore, the memory 420 may include a database system that controls and manages data in a predetermined system. Since the types of memory 420 described above are only examples, the type of memory 420 may be configured in various manners within a range understandable to those skilled in the art based on the content of the present disclosure.

The memory 420 may structure, organize, and manage data, combinations of data, and processor (410)-executable program codes that are required for the processor 410 to perform operations. For example, the memory 420 may store control-related data received via the network unit 430 to be described below.

The network unit 430 according to one embodiment of the present disclosure may be understood as a constituent unit that transmits and receives data through any type of known wired/wireless communication system. For example, the network unit 430 may perform data transmission and reception using a wired/wireless communication system such as a local area network (LAN), a wideband code division multiple access (WCDMA) network, a long term evolution (LTE) network, the wireless broadband Internet (WiBro), a 5th generation mobile communication (5G) network, an ultra-wideband wireless communication network, a ZigBee network, a radio frequency (RF) communication network, a wireless LAN, a wireless fidelity network, a near field communication (NFC) network, or a Bluetooth network. Since the above-described communication systems are only examples, the wired/wireless communication system for the data transmission and reception of the network unit 430 may be applied in various manners other than the above-described examples.

The network unit 430 may receive control-related data required for the processor 410 to perform operations through wired or wireless communication with any system, any client, or the like. Furthermore, the network unit 430 may transmit data generated through the operations of the processor 410 through wired or wireless communication with any system, any client, or the like. The network unit 430 may transmit intermediate data, processed data, and/or the like, derived from the operation process of the processor 410, through communication with the above-described database, server, or computing device, or the like.

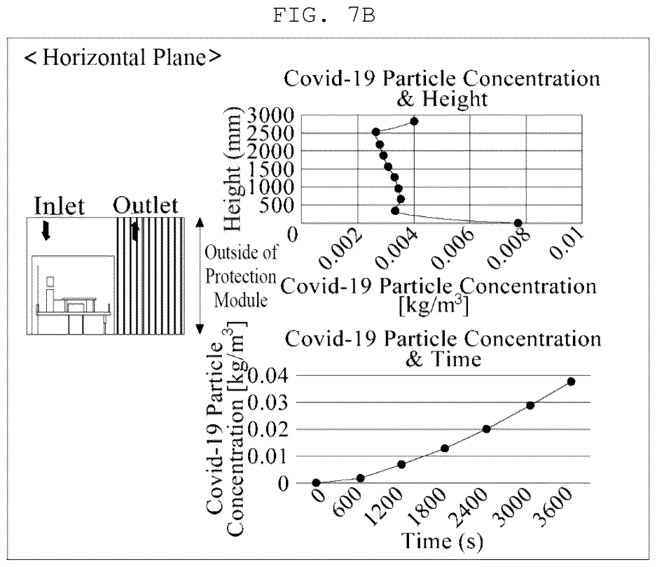

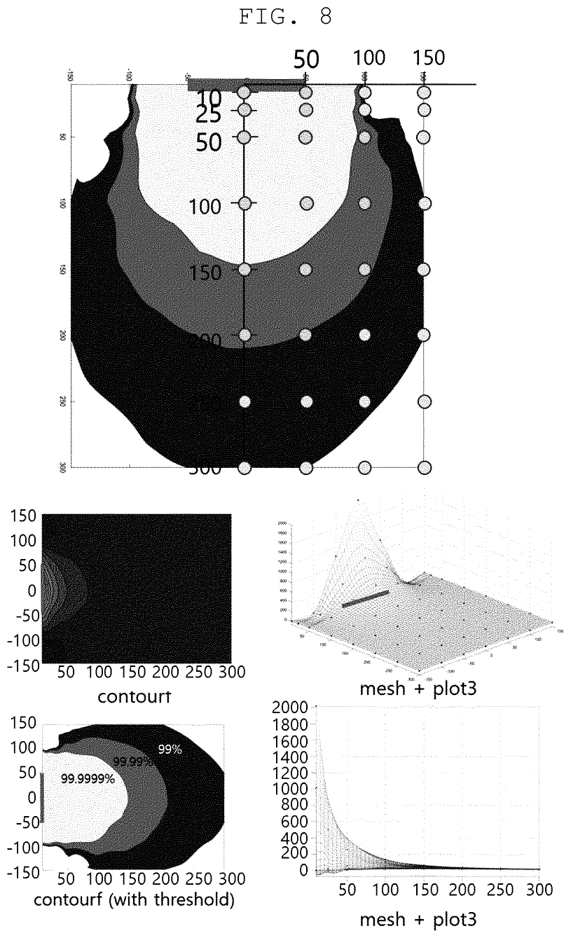

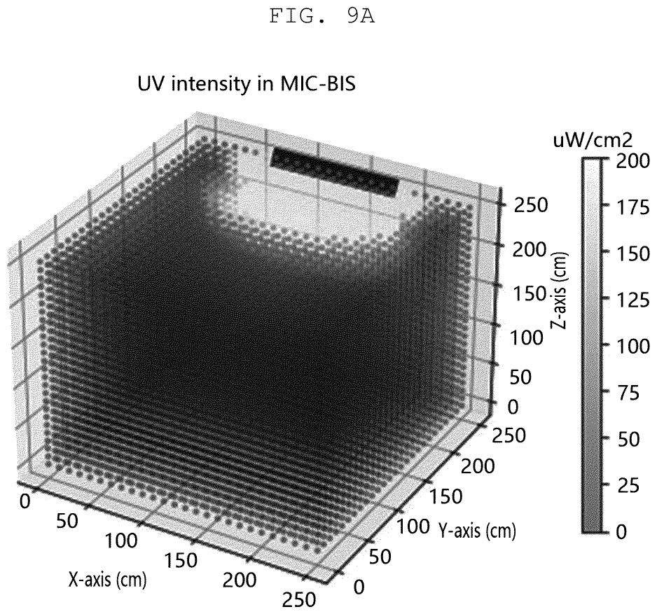

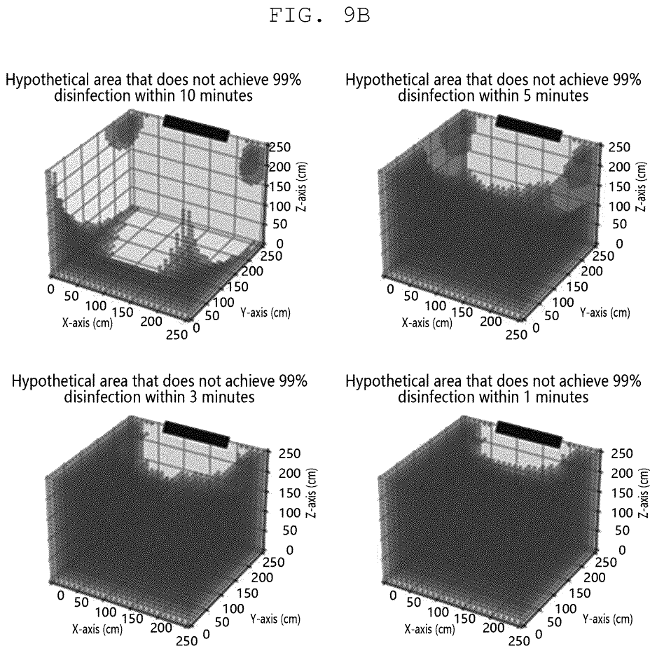

FIG. 5 is a diagram illustrating an infectious agent spread analysis process for selecting the location of an intake according to one embodiment of the present disclosure, FIG. 6 is a diagram illustrating the results of the analysis of a respiratory model through CFD modeling in the infectious agent spread analysis process of FIG. 5, and FIG. 7 is a diagram illustrating the results of the infectious agent spread analysis of FIG. 5.

Referring to FIG. 5, based on the data provided by a medical facility, the computing device may imitate the inside of the protection module 100 as a 3D model and perform infectious agent spread analysis by using Computational Fluid Dynamics (CFD) modeling. In this case, the computing device may be a control server for providing various control services for a simple isolation facility or a medical facility, or an administrator terminal, such as a PC, a laptop, or a personal mobile terminal, while communicating with the control module 400.

Based on the data provided by a medical facility, when the average sitting height of patients aged 20 to 69 is approximately 90 cm and the height D1 of the respiratory organs (noses) is approximately 130 cm from the bed 10, the computing device may derive the results of simulating the respiratory model as shown in FIG. 6 through CFD modeling.

When the average diameter of particles is 5 μm, the shape of the particles is spherical, the density of the particles is 997 kg/m³, and the number of particles is 525, the computing device may represent the particle injection mass flow rate as shown in Equation 1 below:

$$\frac{4}{3} \times π \times {( \frac{5 \times {10}^{- 6}}{2} )}^{3} [ {m}^{3} ] \times 997 [ \frac{kg}{{m}^{3}} ] \times 525 [ \# ] = 3.43 \times {10}^{- 11} [ kg ] \qquad ( 1 )$$

In this case, the volume of the particles (m³), the density of the particles, and the assumed number of the particles are known, so that the computing device may find out that the total mass of the particles is 3.43×10⁻¹¹ kg by using Equation 1 above.

As shown in FIG. 7, the computing device may measure the particle concentration through a virtual plane located outside a passageway area, i.e., the protection module 100, at the time at which simulation is completed for the purpose of infectious agent spread analysis. In this case, the horizontal plane is the density with respect to the height, and the vertical plane is the density with respect to the distance from the protection module 100. The particle mass distribution may be known by measuring the particle mass detected on the patient's body, bed, and floor in the respiratory model, and the negative pressure may be measured by measuring the pressure difference between the inside and outside of the protection module 100.

The computing device may obtain the air circulation rate using Equation 2 below:

$$\begin{matrix}τ = \frac{V}{Q} & {ε}_{v} = \frac{τ}{{θ}_{age}}\end{matrix} \qquad ( 2 )$$

V: Volume of Target Model [m³]
Q: Flow Rate via Inlet [m³/h]
τ: Nominal Ventilation Time [h]
θ_{age}: Age of Air [h]
εᵥ: Ventilation Effectiveness

Referring to FIG. 7, the Covid-19 particle concentration may be analyzed according to the plane height and the simulation time via the horizontal plane, and the results of the particle density analysis in the passageway area show that the concentration is about 10e-6 times lower due to the protection module 100 and the circulation module 200. Using this, the location and area of the intake 121 placed in the ceiling surface of the protection module 100 may be selected to be located vertically above the patient.

The computing device may provide standard facility specifications in consideration of a spatial unit-based infection control requirement level for an emergency medical facility, i.e., the specification requirements of the protection module 100 for the isolation space (the area range, the double structure of the door, the location of the intake, the location and size of the window, the installation locations and number of ultraviolet radiation means, and/or the like), based on the results of the infectious agent spread analysis. The standard facility specifications may maintain the level of the existing emergency patient or infected patient treatment function and maximize the efficiency of infection control. For example, the final location of the fan filter unit 210 of the circulation module 200 may be set by setting various candidate locations, such as the central location of the ceiling surface, the location where the patient's head is located, the location of the entrance (the door), and both side locations based on the center of the isolation space, and checking the degrees of particle spread via CFD modeling.

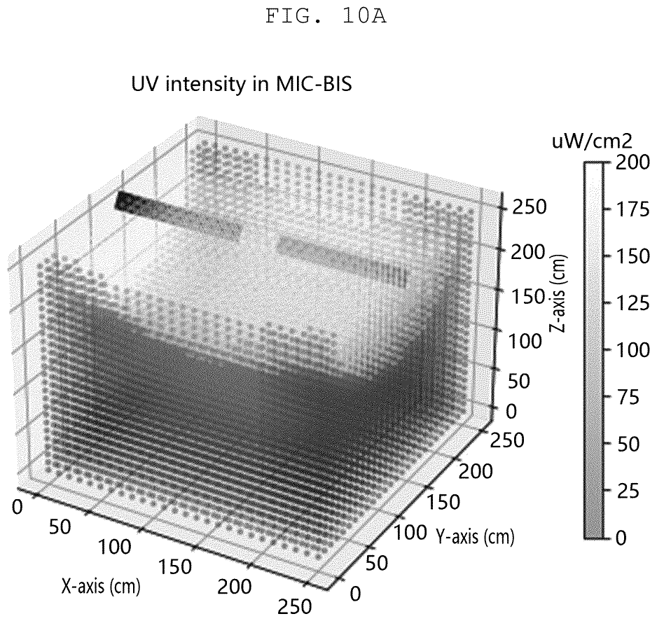

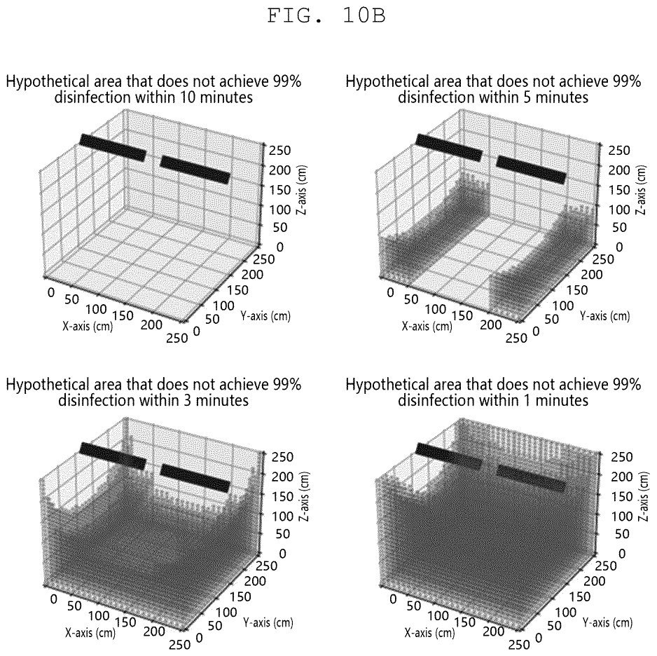

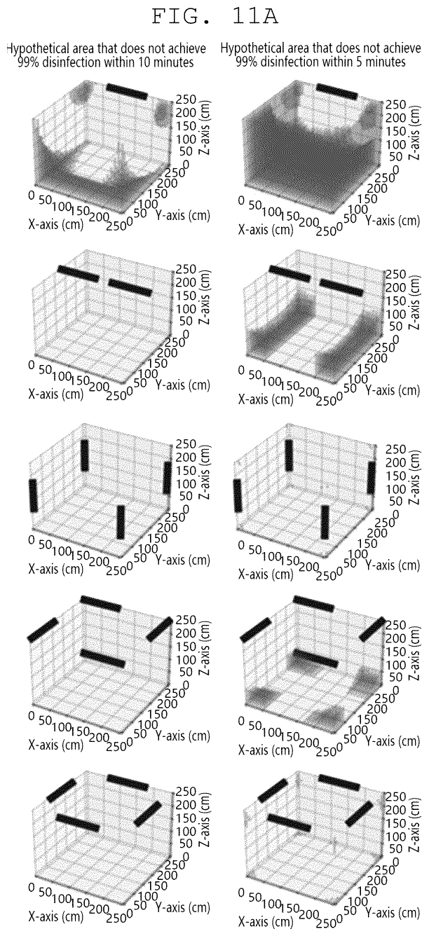

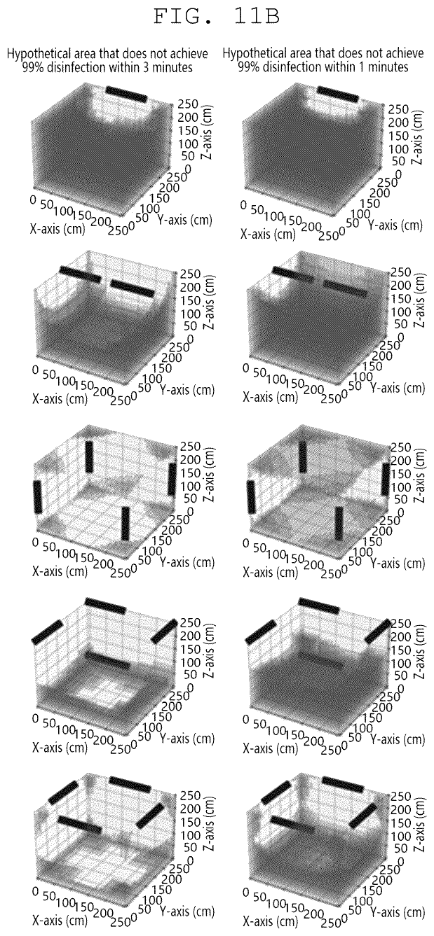

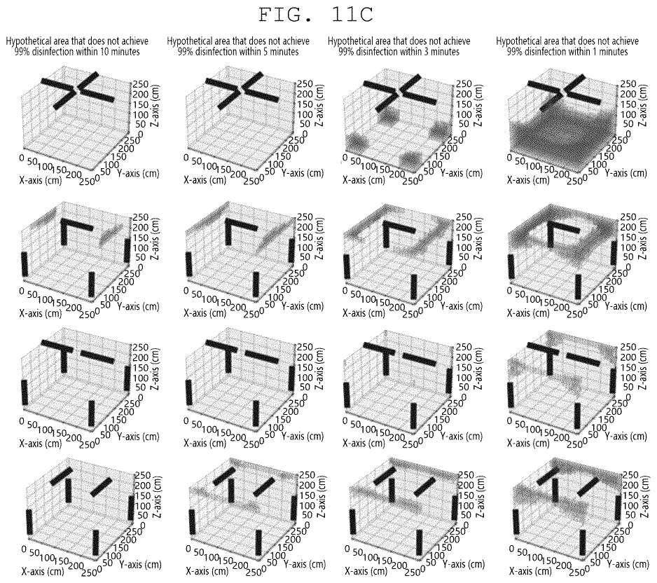

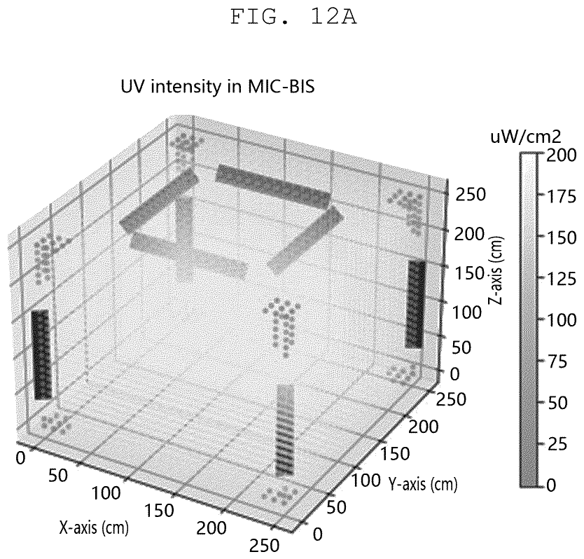

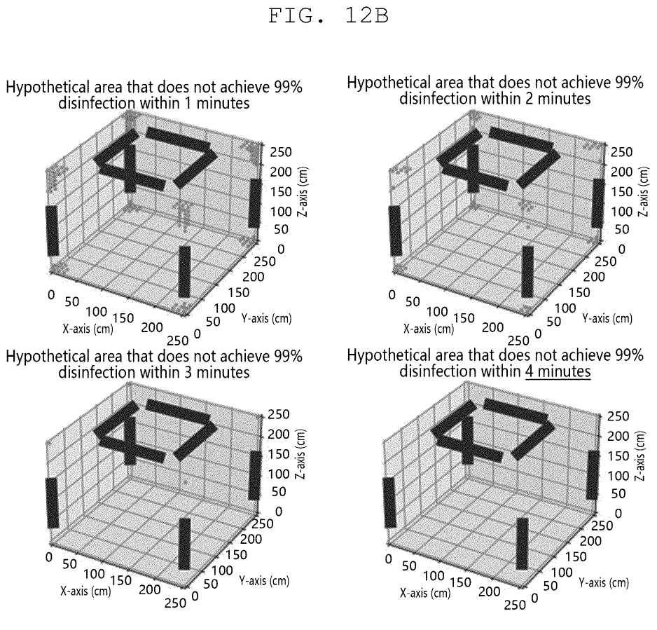

FIG. 8 shows diagrams illustrating an effective disinfection range in a disinfection mode according to one embodiment of the present disclosure, FIG. 9A and FIG. 9B show diagrams illustrating simulation results predicting a time-based disinfection range by means of one ultraviolet radiation means according to one embodiment of the present disclosure, FIG. 10A FIG. show diagrams illustrating simulation results and a time-based disinfection range by means of two predicting ultraviolet radiation means according to one embodiment of the present disclosure, and FIG. 11A, FIG. 11B and FIG. 11C show diagrams illustrating simulation results predicting a time-based disinfection range by means of ultraviolet radiation means arranged at various installation locations according to one embodiment of the present disclosure. FIG. 12A and FIG. 12B show diagrams illustrating an ultraviolet intensity distribution and actually measured ultraviolet intensities according to one embodiment of the present disclosure.

As shown in FIG. 8, the computing device may measure distance-based UV intensities and then check an effective disinfection range by using MATLAB. In practice, as shown in Table 1 below, when the actual measured values of the UV intensities are compared with the effective disinfection range based on an MPSS model, it can be seen that when the distance from a UVC lamp to a disinfection target, i.e., the light radiation distance, is shortened, the light radiation angle also becomes considerably narrower, and accordingly, the effective disinfection range for sterilization and disinfection is significantly reduced. Therefore, it can be seen that in order to expand the light radiation range when the light radiation distance is short, the number of UVC lamps need to be increased.

The computing device may determine the installation locations of UV radiation means based on the simulation results predicting the time-based disinfection range by means of UV radiation means placed at various installation locations, and may set the number of UV radiation means to be placed at each set location to be 4 to 8.

TABLE 1

| Horizontal | | | |
|---|---|---|---|
| Distance | Vertical Distance | Measured Value | Derived Value |
| 0 | 10 | 2248.05 | 2292.964 |
| 0 | 25 | 974.7 | 891.3231 |
| 0 | 50 | 424.85 | 370.5442 |
| 0 | 100 | 145.2 | 119.1922 |
| 0 | 150 | 70.9 | 56.32716 |
| 0 | 200 | 41.575 | 32.42409 |
| 0 | 250 | 27.525 | 20.981 |
| 0 | 300 | 19.275 | 14.65877 |
| 50 | 10 | 1109.75 | 1146.662 |
| 50 | 25 | 519.15 | 458.2869 |
| 50 | 50 | 287.85 | 222.8119 |
| 50 | 100 | 109.3 | 92.6207 |
| 50 | 150 | 56.625 | 49.07076 |
| 50 | 200 | 34.8 | 29.79502 |
| 50 | 250 | 24.4 | 19.82831 |
| 50 | 300 | 17.75 | 14.081 |
| 100 | 10 | 4.625 | 0.182565 |
| 100 | 25 | 25.625 | 12.9882 |
| 100 | 50 | 58.125 | 43.00554 |
| 100 | 100 | 54.375 | 46.63152 |
| 100 | 150 | 38.475 | 33.58166 |
| 100 | 200 | 27.575 | 23.48798 |
| 100 | 250 | 20.025 | 16.8683 |
| 100 | 300 | 15.3 | 12.53358 |
| 150 | 10 | 0 | 0.000115 |
| 150 | 25 | 0 | 0.377555 |
| 150 | 50 | 8.775 | 6.331136 |
| 150 | 100 | 19.35 | 18.78288 |
| 150 | 150 | 20.95 | 19.6213 |
| 150 | 200 | 18.75 | 16.51342 |
| 150 | 250 | 14.825 | 13.17872 |
| 150 | 300 | 12.075 | 10.45354 |

FIGS. 9A to 12B are predictions of a time-based disinfection range based on literature data on the UV intensity and time at which target viruses are disinfected. The simulation code was written in Python and simulation results were obtained using the code. In the drawings, the black dots represent areas that were 5 not disinfected.

First, cubic spline interpolation was performed based on the experimental results of measuring UVC intensity according to horizontal and vertical distances from an UV radiation means when a 100 W UVC lamp was used as the UV radiation means 310.

The UVC intensity value f(v, h) according to the vertical distance v and horizontal distance h from the center of one ultraviolet radiation means 310 may be derived, and the UVC intensity of a point (x, y, z) inside the protection module 100 may be obtained by the sum of the ultraviolet intensities from multiple ultraviolet radiation means 310. In this case, when a multiple points source summation (MPSS) model is applied to the simulation code on the assumption that there is an object (a bed, a chair, and/or the like) inside the isolation space in the disinfection mode, Equation 3 below may be obtained. In Equation 3, P denotes the length of the ultraviolet radiation means 310, n denotes the permittivity, d₁, d₂, and d₃ denote the distances to the points where the ultraviolet intensities from multiple ultraviolet radiation means 310 are measured, respectively, and e denotes the permeability.

$${I}_{i} = ( 1 - R ) \times ( 1 - R ) \times \frac{P / n}{4 {π ( {d}_{1} + {d}_{2} + {d}_{3} )}^{2}} \times {e}^{- α {d}_{2}} \qquad ( 3 )$$

In Equation 3, Snell's law (n₁ sin θ₁=n₂ sin θ₂) and Fresnel's law represented by Equation 4 below are used.

$${R}_{ij} = \frac{1}{2} \{ {( \frac{{n}_{j} cos {θ}_{i} - {n}_{i} cos {θ}_{j}}{{n}_{i} cos {θ}_{j} + {n}_{j} cos {θ}_{i}} )}^{2} + {( \frac{{n}_{i} cos {θ}_{i} - {n}_{j} cos {θ}_{j}}{{n}_{i} cos {θ}_{i} + {n}_{j} cos {θ}_{j}} )}^{2} \} \qquad ( 4 )$$

As a result of the simulation of the MPSS (Multiple points source summation) model, when the dimensions of the isolation space in the protection module 100 are 250 cm*250 cm*250 cm, the intensities of ultraviolet rays in the isolation space may be obtained, and, depending on the operating time (10 minutes, 5 minutes, 3 minutes, or 1 minute) of the ultraviolet radiation means 310, it may be possible to determine the area inside the isolation space where 99% disinfection (10 mJ/cm²) is possible and the area where it is not possible (indicated by the black dots), as shown in FIG. 8.

It can be seen that in the case where there is only one ultraviolet radiation means 310, an ultraviolet intensity of 167 uW/cm² or more is detected when the operation is performed in the disinfection mode within 1 minute, an ultraviolet intensity of 55.7 uW/cm² or more is detected when the operation is performed in the disinfection mode within 3 minutes, an ultraviolet intensity of 33.3 uW/cm² or more is detected when the operation is performed in the disinfection mode within 5 minutes, and an ultraviolet intensity of 16.7 uW/cm² or more is detected when the operation is performed in the disinfection mode within 10 minutes.

In the case where the radius of the lamp of the ultraviolet radiation means 310<<the distance of the UV intensity measurement position (d₁+d₂+d₃≈straight distance) and also in the case where in Snell's law, n₁=1.0 (air), n₂=1.52 (crystal), the transmittance of the crystal e^{-α}=70%/mm, the thickness of the crystal=3 mm, and the length of the point source=1 cm, when the sum of the ultraviolet intensity of each ultraviolet radiation means 310 and the ultraviolet intensities from multiple ultraviolet radiation means 310 is obtained using Equation 3, the sum may be represented by Equation 5 below:

$$I = {\sum }_{1}^{100} ( C \times ( 1 - R ) \times ( 1 - R ) \times \frac{1}{{( {d}_{10} + {d}_{20} + {d}_{30} )}^{2}} \times {0.343}^{1 / cos {θ}_{2}} ) \qquad ( 5 )$$

$$I = C \times {\sum }_{1}^{100} ( ( 1 - R ) \times ( 1 - R ) \times \frac{1}{{( {d}_{10} + {d}_{20} + {d}_{30} )}^{2}} \times {0.343}^{1 / cos {θ}_{2}} )$$

As shown in FIGS. 11 and 12, the computing device may propose the appropriate locations and number of ultraviolet radiation means 310 that control infection to the maximum extent and do not cause side effects to the human body. It can be seen that when the ultraviolet radiation means 310 is placed on the support pillar 111 of the protection module 100 or when the ultraviolet radiation means 310 are placed on the support pillar 111 and the ceiling surface, disinfection may be performed up to a part under the bed 10.

In FIGS. 11A to 11C, the cubic shapes generated using the X axis, the Y axis, and the Z axis each represent a three-dimensional structure for the isolation space, the black bars shown within the cubic shapes represent the ultraviolet radiation means 310 for respective installation locations of the isolation space, and the areas indicated by black dots based on the ultraviolet radiation means 310 represent simulation results predicting the time-based disinfection range by means of the ultraviolet radiation means 310.

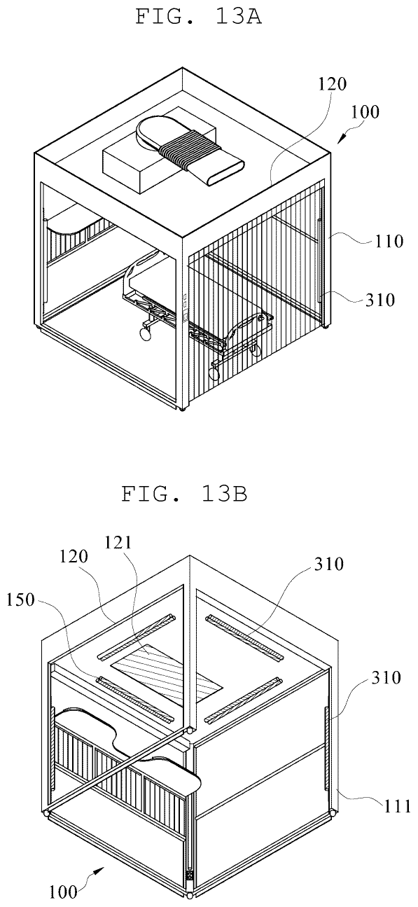

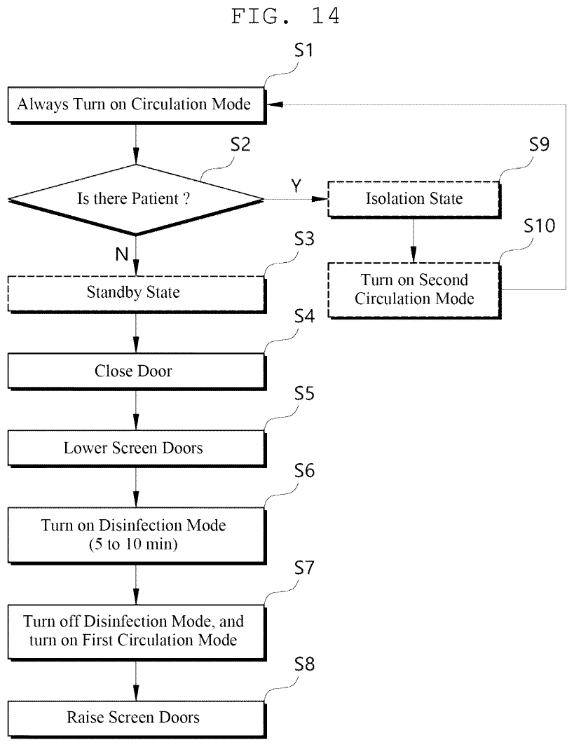

FIG. 13 is a diagram illustrating the installation locations of ultraviolet radiation means according to one embodiment of the present disclosure.

As shown in FIG. 13A, the ultraviolet radiation means 310 may be installed on the respective support columns 111 arranged at the corners of the protection module 100. Furthermore, as shown in FIG. 13B, the ultraviolet radiation means 310 may be additionally arranged on the support columns 111 as well as on the ceiling surface together with the lighting devices 150 arranged at the edges of the ceiling surface at a predetermined distance from the intake 121. In this case, four to eight ultraviolet radiation means 310 may be arranged on the protection module 100.

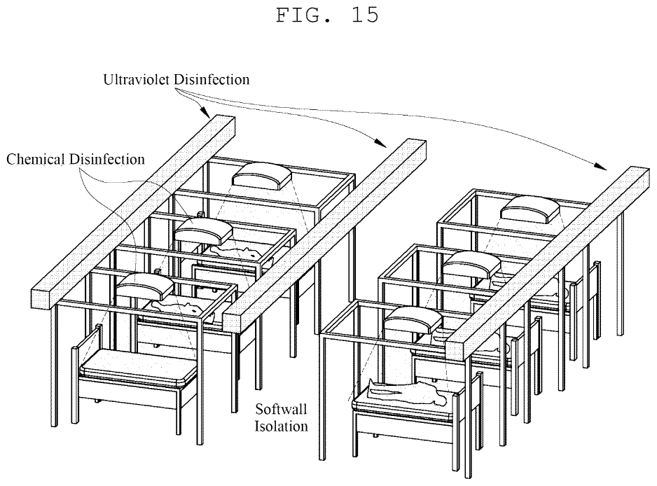

FIG. 14 is a flowchart illustrating a spatial unit-based multimodal infection method for a medical facility according to one embodiment of the present disclosure.

Referring to FIG. 14, the control module 400 ensures that the circulation mode is always in operation by controlling the operation of the circulation module 200 in step S1. The control module 400 checks whether a state in question is a standby state in which there is no patient in the isolation space in steps S2 and S3, closes the strip door 130 in the standby state in step S4, and lowers the screen doors 113 along the outer surfaces of the panels 112 to block the inside of the isolation space, thereby sealing the fronts of the walls 110 so that ultraviolet rays and disinfectant are not exposed to the outside in step S5.

The control module 400 turns on the disinfection mode by operating the ultraviolet radiation means 310 or the disinfectant spray means for a preset operation time (e.g., 5 to 10 minutes) in step S6. When the operation time has elapsed, the disinfection mode is turned off and the first circulation mode is turned on to remove chemical debris formed by the disinfection mode in step S7. The control module 400 raises the screen doors 113 so that the inside of the protection module 100 is visually exposed to the outside in step S8. In this case, by the first circulation mode, the inside of the isolation space may be ventilated, and infectious agents and the harmful components of the disinfectant in the indoor air may be discharged.

Meanwhile, when there is a patient in the isolation space, the control module 400 turns off the disinfection mode so that the disinfection mode is not turned on in the isolation state, and turns on the second circulation mode for performing ventilation and air conditioning for the isolation space to thus block the inflow of outside air or the outflow of inside air in steps S9 and S10.

The control module 400 may set the output of the circulation module 200 in the first circulation mode to be higher than the output of the circulation module 200 in the second circulation mode. The circulation module 200 is always in operation. After the disinfection mode for performing chemical disinfection is completed, the circulation module 200 operates to generate increased output. In contrast, after sufficient treatment is completed, it returns to normal output.

FIG. 15 is a diagram illustrating the configuration of a spatial unit-based multimodal infection control system for a medical facility according to another embodiment of the present disclosure.

As shown in FIG. 15, the system is intended for integrated infection control in the case of a multi-patient room where one or more patients can be accommodated. Soft walls, barriers, or the like, to which vertical laminar flow is applied, are arranged between patient beds by using the transparent panels 112, so that physical contact and air contact that may occur between patients can be blocked. Furthermore, in the system, air leakage is blocked by disposing a circulation module 200 including an intake 121 and a fan filter unit on the top of each of the patient beds.

In the system, a disinfection mode is automatically performed through ultraviolet disinfection and the spraying of disinfectant by using a disinfection module 300 in a standby state in which there is no patient.

In the above description, the vertical laminar flow is a technology that suppresses fine particles to the floor by inducing a constant air flow from top to bottom, and is mainly used in clean rooms for semiconductor processes. According to preceding studies, the size of particles that can contain SARS-CoV-2 is approximately 9.3 μm, so that theoretically, it is impossible for such viruses to move in the air in clean rooms of class 100 to 1000, with the result that it can be seen that aerosol suppression through vertical laminar flow is possible.

The various embodiments of the present disclosure described above may be combined with one or more additional embodiments, and may be changed within the range understandable to those skilled in the art in light of the above detailed description. The embodiments of the present disclosure should be understood as illustrative but not restrictive in all respects. For example, individual components described as unitary may be implemented in a distributed manner, and similarly, the components described as distributed may also be implemented in a combined form. Accordingly, all changes or modifications derived from the meanings and scopes of the claims of the present disclosure and their equivalents should be construed as being included in the scope of the present disclosure.

What is claimed is:

1. A spatial unit-based multimodal infection control system for a medical facility, the spatial unit-based multimodal infection control system comprising:
a protection module including walls configured to form an isolation space, and configured to provide a passage for medical treatment that is disposed in one surface of the walls;
a circulation module disposed in an intake that is formed in a ceiling surface of the walls, and configured to perform ventilation and air-conditioning functions in a circulation mode;
a disinfection module including at least one disinfection source placed on an inner wall surface or ceiling surface of the walls, and configured to disinfect an inside of the isolation space by using the disinfection source in a disinfection mode; and
a control module configured to control the disinfection module and the circulation module so that the disinfection mode and the circulation mode are turned on in a standby state in which there is no patient in the isolation space.

2. The spatial unit-based multimodal infection control system of claim 1, wherein the protection module comprises:
the walls arranged vertically based on a floor surface, and configured to provide the isolation space;
a ceiling panel disposed on the ceiling surface located over the walls, and configured to close the isolation space except for the intake;
a strip door disposed in the passage; and
a window disposed in one surface of the walls located in a direction facing the strip door, and serving as a passage for entry and exit of a wiring means for connection with a medical device or power source provided in a medical facility.

3. The spatial unit-based multimodal infection control system of claim 2, wherein the walls comprise:
one or more support columns vertically arranged at respective corners of the isolation space;
panels arranged between the individual support columns, and forming wall surfaces except for the wall surface in which the strip door is installed; and
screen doors configured to be selectively opened and closed in vertical directions along outer surfaces of the walls by sliding rollers arranged at top ends of the walls.

4. The spatial unit-based multimodal infection control system of claim 3, wherein the control module controls the screen doors to be closed in order to cover the inside of the isolation space in the disinfection mode.

5. The spatial unit-based multimodal infection control system of claim 1, wherein the circulation module comprises:
a fan filter unit configured to perform internal ventilation and air-conditioning functions by blocking an inflow of outside air and an outflow of inside air to and from the isolation space; and
an adaptive nozzle disposed to discharge an air flow, formed by the fan filter unit, out of the isolation space, and formed to be rotatable in order to operate in conjunction with an air-conditioning system of the medical facility.

6. The spatial unit-based multimodal infection control system of claim 5, wherein the fan filter unit includes at least one of a HEPA filter, an activated charcoal filter, and a pre-filter.

7. The spatial unit-based multimodal infection control system of claim 1, wherein the disinfection source comprises at least one of one or more ultraviolet radiation means and one or more disinfectant spray means.

8. The spatial unit-based multimodal infection control system of claim 7, wherein installation locations and a number of the ultraviolet radiation means are set based on a predicted time-based effective disinfection range, and the time-based effective disinfection range in which the isolation space is disinfected is predicted based on an ultraviolet intensity by means of the ultraviolet radiation means and operation time information of the disinfection mode.

9. The spatial unit-based multimodal infection control system of claim 8, wherein the time-based effective disinfection range is calculated as a time-based effective disinfection range based on the installation locations and number of the ultraviolet radiation means according to a simulation using a multiple points source summation (MPSS) model in a case where there is an object inside the isolation space.

10. The spatial unit-based multimodal infection control system of claim 9, wherein the ultraviolet intensity (Iᵢ) by means of the MPSS model is calculated by Equation 1 below using Snell's law and a Fresnel coefficient, and the ultraviolet intensity by means of n ultraviolet radiation means is calculated by summing all ultraviolet intensities by means of the respective ultraviolet radiation means disposed at different installation locations:
$${I}_{i} = ( 1 - R ) \times ( 1 - R ) \times \frac{P / n}{4 {π ( {d}_{1} + {d}_{2} + {d}_{3} )}^{2}} \times {e}^{- α {d}_{2}} \qquad ( 1 )$$
P: length of the ultraviolet radiation means
n: permittivity
d₁, d₂, and d₃: distances to points where the ultraviolet intensities from the individual ultraviolet radiation means are measured, respectively
$$Fresnel coefficient : {R}_{ij} = \frac{1}{2} \{ {( \frac{{n}_{j} cos {θ}_{i} - {n}_{i} cos {θ}_{j}}{{n}_{i} cos {θ}_{j} + {n}_{j} cos {θ}_{i}} )}^{2} + {( \frac{{n}_{i} cos {θ}_{i} - {n}_{j} cos {θ}_{j}}{{n}_{i} cos {θ}_{i} + {n}_{j} cos {θ}_{j}} )}^{2} \}$$
Snell's law: n₁ sin θ₁=n₂ sin θ₂
e: permeability

11. The spatial unit-based multimodal infection control system of claim 9, wherein the ultraviolet radiation means comprises at least one ultraviolet radiation means installed at each corner of the isolation space, or at least one ultraviolet radiation means installed on an edge side of the ceiling surface.

12. The spatial unit-based multimodal infection control system of claim 1, wherein:
the spatial unit-based multimodal infection control system further comprises a motion detection means disposed on one side of the passage; and
the control module controls an ON/OFF operation of the disinfection mode or an ON/OFF operation of a lighting device disposed in the protection module based on a detection result of the motion detection means.

13. A spatial unit-based multimodal infection control method for a medical facility, the spatial unit-based multimodal infection control method being performed by a system including at least one processor, the spatial unit-based multimodal infection control method comprising:
turning on a disinfection mode that performs disinfection by means of a disinfection source for a preset operation time in a standby state in which there is no patient inside a protection module that forms an isolation space by using walls;
turning off the disinfection mode after the operation time has elapsed in the standby state, and turning on a first circulation mode that removes chemical debris formed by the disinfection mode; and
turning off the disinfection mode and turning on a second circulation mode that performs ventilation and air conditioning functions for the isolation space in an isolation state in which there is a patient in the isolation space;
wherein the disinfection source comprises at least one of one or more ultraviolet irradiation means and one or more disinfectant spray means.

14. The spatial unit-based multimodal infection control method of claim 13, wherein an output of a circulation module in the first circulation mode is set to be higher than that of the circulation module in the second circulation mode.
